(19) United States
(12) Patent Application Publication
(10) Pub. No.: US 2017/0231819 A1
(43) Pub. Date: Aug. 17, 2017

(54) VISION CORRECTION APPARATUS AND METHOD FOR CONTROLLING SAME

(71) Applicant: Lutronic Corporation, Gyeonggi-do (KR)

(72) Inventor: Hee Chul Lee, Gyeonggi-do (KR)

(21) Appl. No.: 15/583,770

(22) Filed: May 1, 2017

Related U.S. Application Data
(63) Continuation-in-part of application No. 14/360,510, filed on May 23, 2014, now abandoned.

(30) Foreign Application Priority Data
Nov. 24, 2011 (KR) 10-2011-0123562

(51) Int. Cl. A61F 9/008; A61F 9/013; A61B 18/20

(52) U.S. Cl. CPC A61F 9/00836; A61B 18/20; A61F 9/00802; A61F 9/013; A61B 2018/00619

(57) ABSTRACT

The vision correction apparatus according to the present invention comprises: a cutting-off beam generating unit that generates a cutting-off beam for cutting off a part of the cornea for a vision correction surgery, a welding beam generating unit that generates a welding beam having a wavelength in a near-infrared band for welding a part of the cut cornea by irradiating the welding beam to the cut position of the cornea, a beam delivery unit that delivers the cutting-off beam and the welding beam to the cut position of the cornea, an image unit that obtains image information on the cut position of the cornea, and a control unit that controls an irradiation position of the welding beam based on the cut position information of the cornea obtained by the image unit.

### RELATED APPLICATIONS

This application is a continuation-in-part of and claims priority to U.S. Patent Application No. 14/360,510 filed May 23, 2014, which is a 35 U.S.C. §371 National Stage Entry of PCT/KR2012/010075 filed Nov. 26, 2012, which claims priority to Korean Patent Application 10-2011-0123562 filed Nov. 24, 2011. The entire contents of each of the foregoing applications are incorporated herein by reference in their entirety.

### FIELD OF THE INVENTION

The present invention relates to a vision correction apparatus and a method of controlling the same, and more particularly a vision correction apparatus that corrects vision, using a laser and a method of controlling the vision correction apparatus. Background

Recently vision correction apparatuses that satisfy various types of operations for correcting vision have been developed. In addition to vision correction apparatuses that can satisfy the operation types, for example, LASIK (laser in situ keratomileusis) and LASEK (laser assisted sub-epithelial keratomileusis), vision correction apparatuses that can perform various operations such as wavefront LASIK, epi-LASIK, and i-LASIK, which are included in LASIK, have been developed.

The vision correction apparatus performing an operation in the type of LASIK in the vision correction apparatuses is characterized in that it creates a flap by cutting off a portion of a cornea and radiating a vision correction beam to the stromal bed that is the portion with the flap separated, thereby correcting vision. The separated flap is arranged to cover the stromal bed after vision correction.

As a vision correction apparatus of the related art, there is a “device for separation of corneal epithelium” disclosed in Korean Patent Application Publication No. 2006-0097709. The prior art document provides a device for the LASIK surgery, which includes a handpiece having a traverse motor and a vibrator motor for creating a flap separated from a portion of a cornea.

However, the device has a problem in that it uses a handpiece for cutting off a portion of a cornea to perform the LASIK surgery, but the flap separated by the handpiece is arranged simply cover the stromal bed, such that the flap may be separated, when impact is applied the cornea.

### SUMMARY OF THE INVENTION

The present invention has been made in an effort to provide a vision correction apparatus that improves the type of a vision correction operation so that a flap separated from a cornea can be permanently supported on the cornea, and a method of controlling the vision correction apparatus.

A vision correction apparatus according to the present invention includes: a cutting-off unit that cuts off a portion of a cornea to provide a stromal bed to be corrected and a flap partially separated from the cornea; an image unit that collects and processes images about states between the cornea and the flap positioned over the stromal bed and covering the stromal bed after the stromal bed is corrected; a beam generating unit that generates a welding beam for welding the cut portion between the flap and the cornea on the basis of an image signal processed by the image unit; a beam delivery unit that guides the welding beam generated by the beam generating unit along the cut portion of the flap and the cornea; and a control unit that controls the operation of the beam delivery unit so that the welding beam is radiated along the cut portion between the flap and the cornea, on the basis of the image signal processed by the image unit.

The image unit may include: an image collecting part that collects images about the cutting status of the cornea when the cornea is cut off, and collects image about the welding status when the cornea and the flap are welded; and an image processing part that processes images from the image collecting part and transmit them to the control unit.

Preferably, the image unit may include an optical coherent tomography.

The cutting-off unit may include a microkeratome.

On the other hand, it is preferable that the cutting-off unit radiates a femtosecond laser to the cornea.

The welding beam generated by the beam generating unit may include a femtosecond laser.

The vision correction apparatus may further include an objective lens that is disposed between the cornea and the beam generating unit and concentrates the welding beam generated by the beam generating unit.

Further, the vision correction apparatus may further include a regulating unit that regulates the distance between the cornea and the objective lens.

A method of controlling a vision correction apparatus according to the present invention includes: (a) cutting off a portion of a cornea to create a flap that is a portion separated from the cornea; (b)covering a stromal bed with the flap and radiating a welding beam to the cut portion of the cornea and the flap, after the stromal bed with the flap separated is corrected; and (c) controlling the radiation position of the welding beam so that the welding beam is radiated along the cut portion of the cornea and the flap, when the cornea and the flap are welded.

The step (c) may include collecting and processing images about the welding status of the cornea and the flap and the radiation position of the welding beam, when the cornea and the flap are welded.

Preferably, the step (a) may use any one of a microkeratome and a femtosecond laser.

The vision correction apparatus may include an objective lens that concentrates the welding beam radiated to the cornea.

The step (b) may include regulating the distance between the cornea and the objective lens.

The welding beam may include a femtosecond laser.

The details of other embodiments are included in the following detailed description and the accompanying drawings.

According to the vision correction apparatus and a method of controlling the vision correction apparatus of the present invention, it is possible to prevent the flam from being separated by an external impact by welding the cut portion between the cornea and the flap separated from the cornea with the welding beam, and thus it is possible to the patient's satisfaction at the operation.

### BRIEF DESCRIPTION OF THE DRAWINGS

FIG. 1 is a control block diagram of a schematic configuration of a vision correction apparatus according to an embodiment of the present invention.

FIGS. 2A to 2C are perspective views illustrating the operation of a vision correction apparatus according to a first embodiment of the present invention.

FIGS. 3A to 3C are perspective views illustrating the operation of a vision correction apparatus according to a second embodiment of the present invention.

FIG. 4 is a flowchart illustrating control of the vision correction apparatuses according to the first and second embodiments of the present invention.

FIG. 5 is a graph illustrating an absorption characteristic according to a wavelength of a beam.

FIG. 6 is a cross-sectional view illustrating an appearance in which a welding beam is irradiated to a cut position of the cornea.

FIG. 7 is a block diagram of a vision correction apparatus according to a third embodiment of the present invention.

FIG. 8 is a diagram illustrating an example of a pattern irradiated with the welding beam.

### DESCRIPTION OF EXEMPLARY EMBODIMENTS

Hereinafter, vision correction apparatuses according to embodiments of the present invention and a method of controlling the vision correction apparatuses are described in detail with reference to the accompanying drawings.

Before the description, different vision correction apparatuses of the present invention will be described in the first and second embodiments, but it should be understood that the components in the configuration of the vision correction apparatuses according to the first and second embodiments are given the same names and reference numerals.

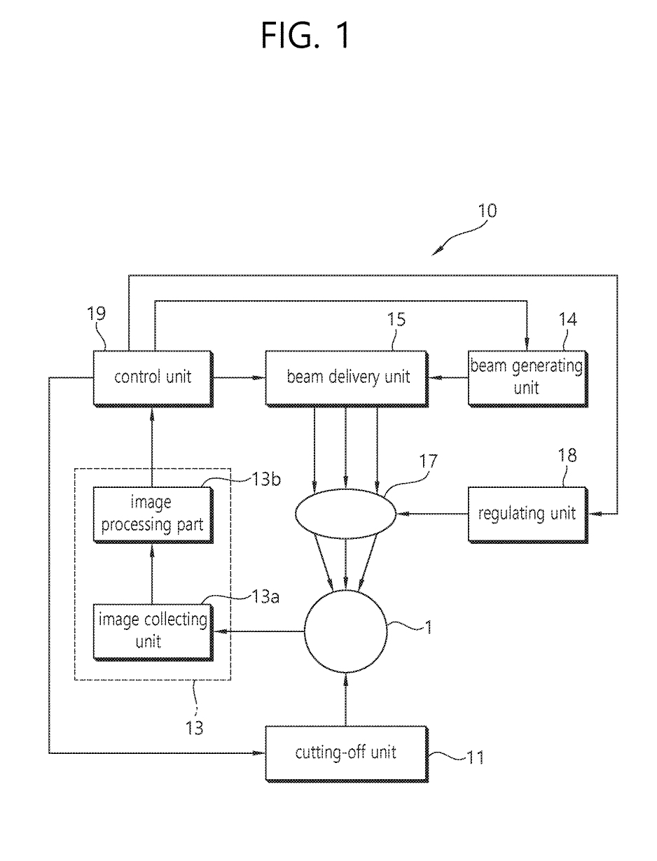

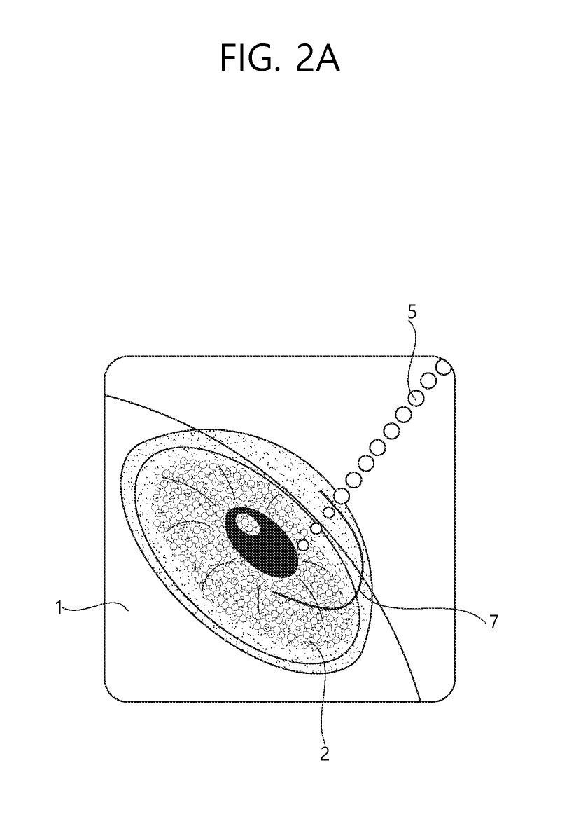

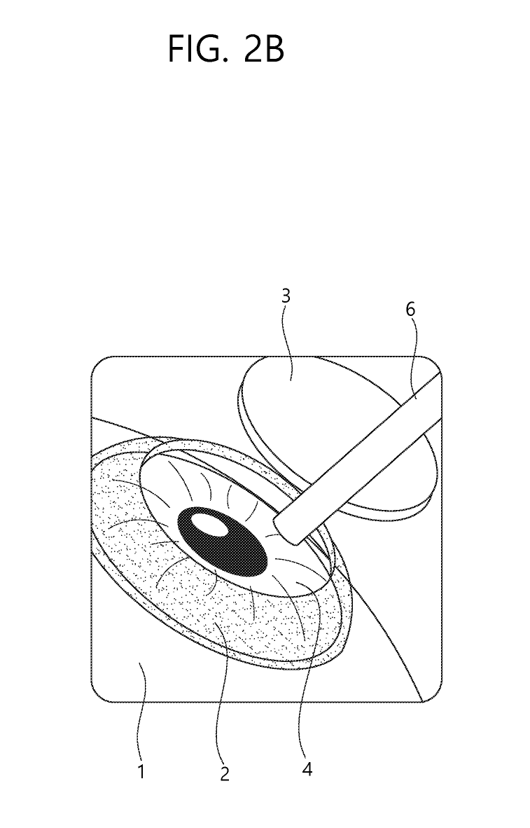

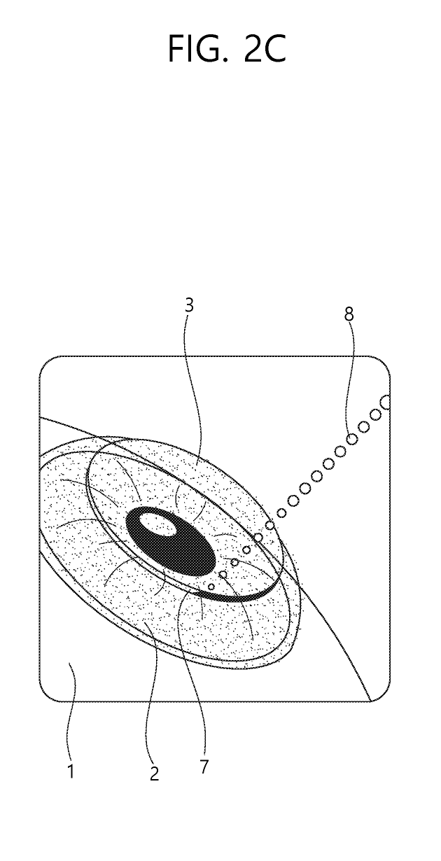

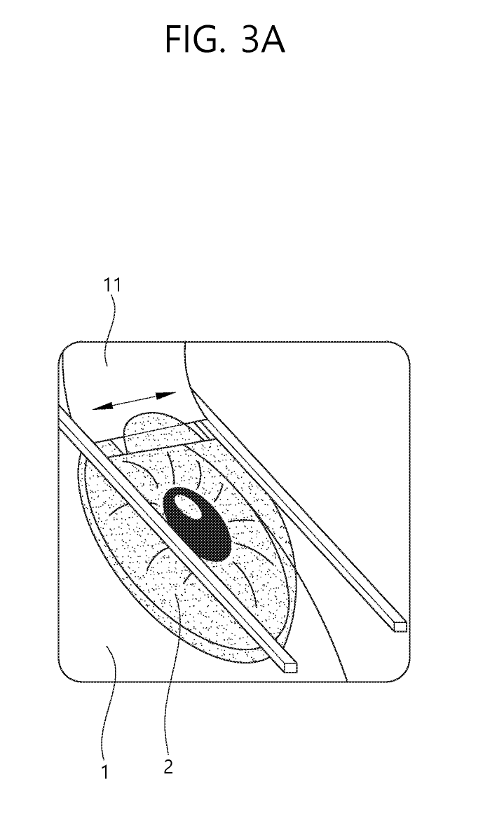

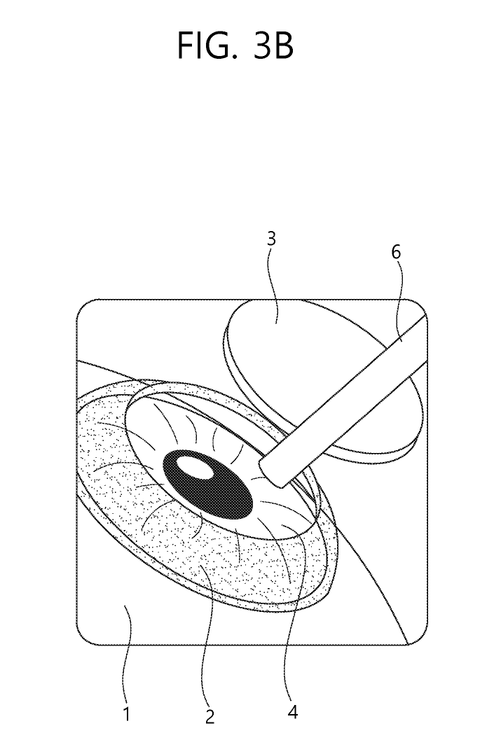

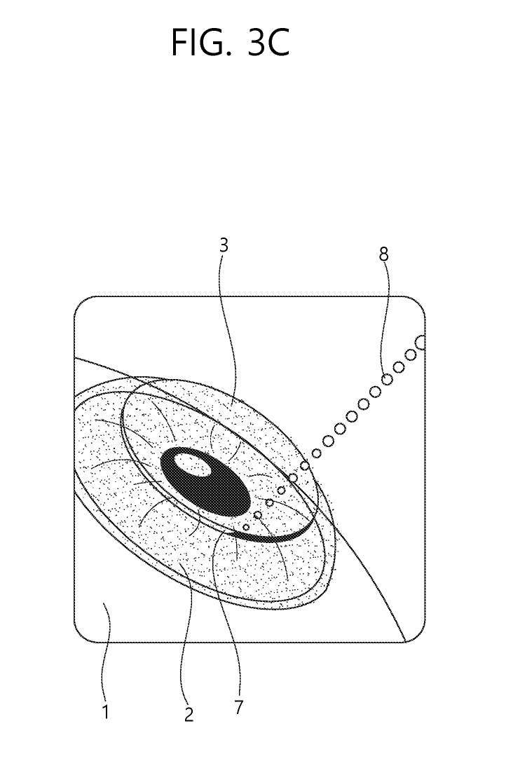

FIG. 1 is a control block diagram of a schematic configuration of a vision correction apparatus according to an embodiment of the present invention, FIGS. 2A to 2C are perspective views illustrating the operation of a vision correction apparatus according to a first embodiment of the present invention, and FIGS. 3A to 3C are perspective views illustrating the operation of a vision correction apparatus according to a second embodiment of the present invention.

As shown in FIGS. 1 to 3C, a vision correction apparatus 10 according to an embodiment of the present invention includes a body (not shown), a cutting-off unit 11, an image unit 13, a beam generating unit 14, a beam delivery unit 15, an objective lens 17, a regulating unit 18, and a control unit 19. The vision correction apparatus 10 according to an embodiment of the present invention is used for a LASIK (laser in-situ keratomileusis) surgery.

The body forms the external shape of the vision correction apparatus 10 and receives or is equipped with the cutting-off unit 11, the image unit 13, the beam generating unit 14, the beam delivery unit 15, the objective lens 17, the regulating unit 18, and the control unit 19.

The cutting-off unit 11 cuts off a portion of a cornea 2 so that a vision correction beam 6 can be radiated to the stromal bed 4, that is, the stroma of the cornea. A flap 3, which is the portion of the cornea 2 cut off by the cutting-off unit 11 is cut off from the cornea 2. The flap 3 cut off by the cutting-off unit 11 is not completely separated from the cornea 2, but connected to a portion of the cornea 1 through a small area. The flap 3 formed by the cutting-off unit 11 has an epithelium and a bowman's membrane.

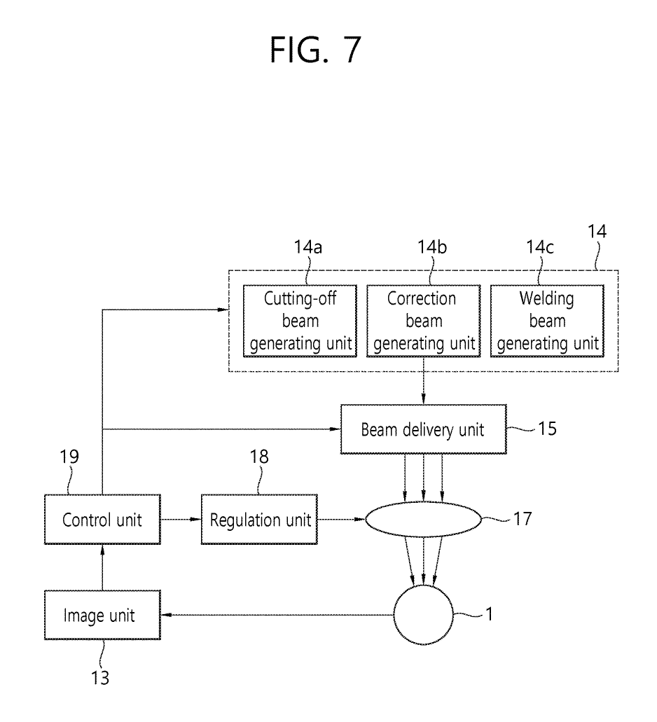

The cutting-off unit 11 according to the first embodiment of the present invention cuts off the cornea 2, using a femtosecond laser that is a cutting-off beam 5. That is, as shown in FIG. 2A, the cutting-off unit 11 cuts off the cornea 2 to create the flap 3 that is separated from the cornea 2 by radiating a femtosecond laser in a closed loop shape to an area on the cornea 2. The cutting-off unit 11 according to the second embodiment of the present invention is a part independent from the beam generating unit 14 in FIG. 1, but it may be integrated with the beam generating unit 14 (see FIG. 7).

The cutting-off unit 11 according to the second embodiment of the present invention is microkeratome using a blade to cut off the cornea 2. That is, as shown in FIG. 3A, the cutting-off unit 11 that is a microkeratome is moved over the cornea 2 and cuts off a portion of the cornea 2 to create the flap 3 to be separated from the cornea 2.

The vision correction apparatuses according to the first and second embodiments of the present invention, as shown in FIGS. 2B and 3B, radiate the vision correction beam 6 to the stromal bed 4 formed by the cutting-off unit 11. The configuration that radiates the vision correction beam 6 may be integrated with the beam generating unit 14 that radiates a welding beam 8 (see FIG. 7) or may be provided independently from the beam generating unit 14.

Next, the image unit 13 collects and processes image so that the operation state of an eyeball 1 can be monitored, when the cutting-off unit 11 cuts off the cornea 2 and the beam generating unit 4 and the beam delivery unit 15 are operated to weld the cut portion 7 of the flap 3 to the cornea 2. For example, the image unit 13 collects and processes images about the cutting depth and the cutting range of the cornea 2, when the cornea 2 is cut off, and it collects and processes an image about the welding status of the cut portion 7, when the flap 3 is welded to the cornea 2.

The image unit 13 of the present invention includes an image collecting part 13a that collects images of the eyeball 1 and an image processing part 13b that processes the images collected by the image collecting part 13a. The processing signals by the image processing part 13b are transmitted to the control unit 19 so that the beam generating unit 14 and the beam delivery unit 15 can be controlled.

The image unit 13 of the present invention includes an OCT (Optical Coherence Tomography). The OCT that is the image unit 13 can measure distances from the wavelengths of light reflecting from different structures of the eyeball 1, using short coherent light, and collect and processes high-resolution transverse images. Thee image unit 13 that is an OCT can induce a more precise operation by collecting and processing images in real time with cutting-off and welding of the cornea 2 and transmitting them to the control unit 19.

The beam generating unit 14 generates a welding beam 8 for welding the cut portion 7 between the cornea 2 and the flap 3 on the basis of the image signals processed by the image unit 13, when the cornea 2 and the flap 3 are welded. The welding beam 8 generated by the beam generating unit 14 is a femtosecond laser.

The wavelength of the welding beam 8 uses a band having a high absorption in the cornea 2. In this case, when the wavelength band of the welding beam 8 is too high, a large amount of absorption occurs on the surface of the cornea 2, and thus, in order to obtain the required result, a wavelength band having an appropriate absorption rate needs to be selected. Further, in the case of a pulse width of the welding beam 8, when considering a thermal relaxation time (TRT) of the corneal tissue, a pulse width longer than the TRT needs not to be irradiated, and thus, a pulse width shorter than the TRT is required.

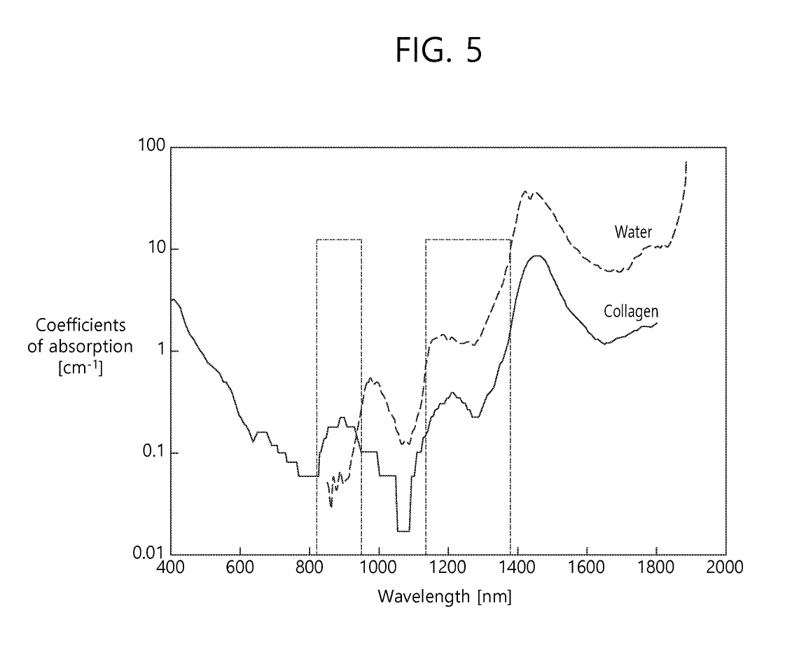

FIG. 5 is a graph illustrating an absorption characteristic according to a wavelength of a beam. As illustrated in FIG. 5, an absorption characteristic varies according to a wavelength of a beam. For example, the absorption characteristic of the beam to collagen is decreased as the wavelength is increased in a visible light area, and the absorption characteristic tends to be increased as the wavelength is increased in a wavelength of 1000 nm or more. In addition, the absorption characteristic of the beam to water tends to be increased as the wavelength is increased in a wavelength of 1000 nm or more.

As such, as a result of experiments on various wavelengths having different absorption characteristics, it is determined that in the case of using a beam in a near-infrared band having a good absorption characteristic to collagen as the welding beam, it is advantageous in welding of the cornea. However, when the absorption characteristic to water is too high, there is a problem such as a phenomenon in which the beam is absorbed in the corneal tissue before reaching the corneal welding position. Accordingly, even though the absorption characteristic to collagen is excellent, the beam of 1400 nm or more having a high absorption characteristic to water may damage the surface tissue.

Considering this, in the present embodiment, a beam in a near-infrared wavelength band having an absorption coefficient to collagen of 0.1 to 1 may be used as the welding beam. As illustrated in FIG. 5, the wavelength of the welding beam may be in a range of 800 nm to 980 nm or 1150 nm to 1390 nm. More particularly, the wavelength of the welding beam may be in a range of 840 nm to 950 nm or 1160 nm to 1370 nm. In this case, while thermal damage to a corneal surface tissue or adjacent tissue is minimized, a corneal flap may be welded by transferring energy to the welding position. Therefore, the corneal flap can be welded by the welding beam without using additional bonding material or photo-activated material.

The beam delivery unit 15 guides the welding beam 8 generated by the beam generating unit 14 along the cut portion of the cornea 2 and the flap 3, as shown in FIGS. 2C and 3C. The beam delivery unit 15 includes a scanner that can adjust the radiation position of the welding beam 8 generated by the beam generating unit 14. The beam delivery unit 15 is controlled by the control unit 19 such that the welding beam 8 is radiated to appropriate positions along the cut portion 7 of the cornea 2 and the flap 3 or in accordance with the cutting depth.

The beam delivery unit 15 includes an objective lens 17 provided at the end of the path through which the beam is irradiated. The objective lens 17 is provided between the beam generating unit 14 and the cornea 2 to collect the beam delivered through the beam delivery unit 15. As a result, a spot size of the welding beam 8 irradiated to the cut position may be a size of 0.1 μm to 10 μm. The welding beam is irradiated to the cut position between the cornea 2 and the flap 3 to increase up to a temperature at which the tissue at the cut position is denatured and weld the cornea 2 and the flap 3 to each other. The regulating unit 18 is provided to regulate the distance between the objective lens 17 and the cornea 2. The regulating unit 18 may be a lens barrel such as a camera or other parts such as a motor known in the art. The regulating unit 18 regulates the distance between the cornea 2 and the objective lens 17 so that the cornea 2 and the flap 3 can be easily welded.

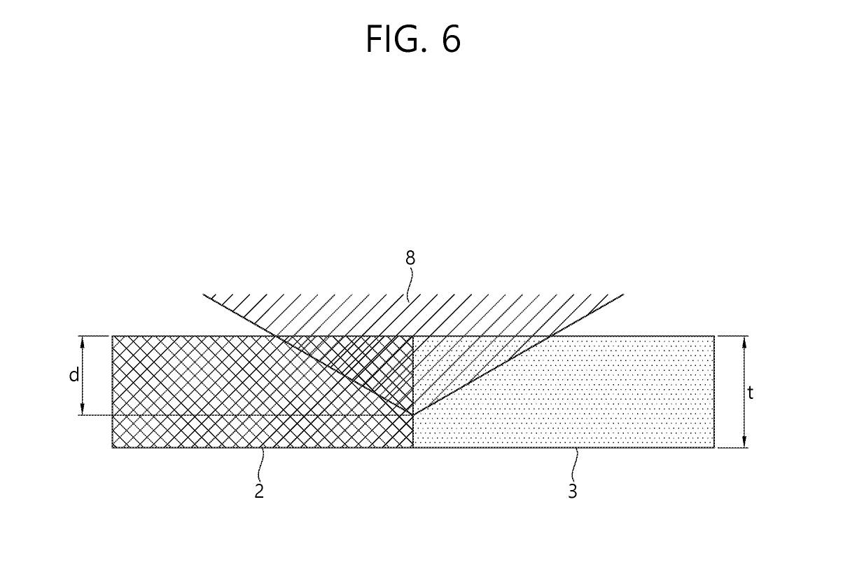

Hereinafter, an appearance in which the welding beam is irradiated to the cut position of the cornea by the beam delivery unit will be described in detail with reference to FIG. 6.

One side of the lesion illustrated in FIG. 6 is a non-cut cornea and the other side is a portion forming a flap when cutting off the cornea, and for convenience of description, it is illustrated as a straight line rather than a curved surface. As illustrated in FIG. 6, the welding beam is delivered by the beam delivery unit to be irradiated to the cut position corresponding to the edge of the corneal flap. In this case, a target position d where the welding beam is focused may be positioned at an inner side with a predetermined depth from the surface of the cut position. The welding is performed at the inner side with the predetermined depth of the cut position to minimize degeneration of the corneal surface and improve a welding characteristic of the cornea. In this case, a depth d of the target position where the welding beam is focused may have a size of 20% to 80% of a flap thickness t formed by cutting off the cornea.

In addition, as illustrated in FIG. 6, the welding beam irradiated by the beam delivery unit 15 is converged to be focused to the target position. In the present embodiment, an angle of the welding beam which is converged to the cornea through the objective lens 17 may be in a range of 10° to 60°. As a result, it is possible to concentrate energy to the target position while minimizing damage to the corneal surface.

Meanwhile, in FIG. 1, it is illustrated that the beam generating unit 14 generates the welding beam and the beam delivery unit 15 delivers only the welding beam. However, as described above, the cutting-off beam and the vision correction beam are integrated and provided at the beam generating unit and the respective beams may be delivered by using one beam delivery unit. As illustrated in FIG. 7, the beam generating unit may be configured by including a cutting-off beam generating unit, a correction beam generating unit, and a welding beam generating unit. In addition, the beam delivery unit is configured to deliver the cutting-off beam, the correction beam, and the welding beam along a common path, respectively, to deliver light required depending on a surgery stage to the cornea. In this case, the cutting-off beam, the correction beam, and the welding beam may be configured by light with the same wavelength, but may be configured by using light having different wavelengths by considering the roles. In addition, as compared with the correction beam and the welding beam, the cutting-off beam may be configured to have a high output to deliver high energy per unit area of the cornea.

Referring back to FIG. 1, the control unit 19 controls the operation of the beam delivery unit 15 to adjust the radiation position of the welding beam 8 radiated to the cut portion 7 between the cornea 2 and the flap 3 on the basis of signals from the image unit 13. That is, the control unit 19 controls the operation of the beam delivery unit 15 that can change the radiation path of the welding beam 8 so that the welding beam 8 is radiated along the cut portion 7 between the cornea 2 and the flap 3 by analyzing image signals transmitted in real time from the image unit 13. The control unit 19 can control the cutting-off unit 11 that cuts off the cornea 2, on the basis of image signals from the image unit 13, in an operation of cutting off the cornea 2.

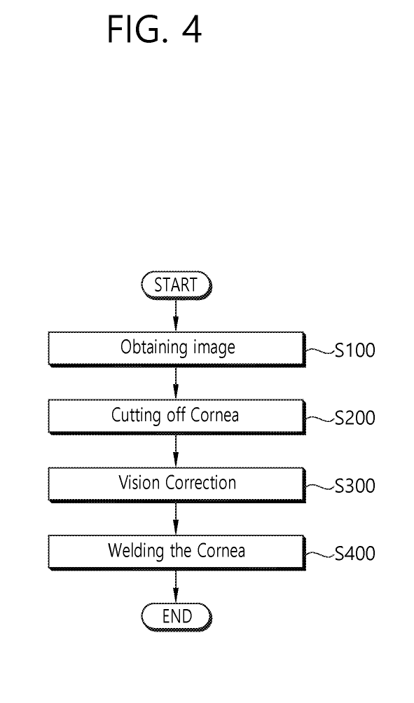

FIG. 4 is a flowchart illustrating control of the vision correction apparatuses 10 according to the first and second embodiments of the present invention.

A method of controlling the vision correction apparatuses 10 according to the first and second embodiments of the present invention which have the configurations described above are described hereafter with reference to FIG. 4.

First, a step of obtaining the image of the cornea for performing an ophthalmic surgery is performed (S100). The present step is performed by using the aforementioned image unit 13 and the in the present embodiment, the image of the cornea including topographic information of the cornea may be obtained by using an OCT apparatus. A user may design the surgery content including the cut position of the cornea based on the image information of the cornea obtained in the present step.

Next, a step of cutting off the cornea of a patient is performed to perform a vision correction surgery (S200). In the step, the control unit drives the cutting-off beam generating unit to generate the cutting-off beam and operates the beam delivery unit to irradiate the cutting-off beam to the cut position of the cornea. A part of the cornea cut off by the irradiated cutting-off beam may form a flap separated from the cornea of the patient. However, as an example, the part can be cut off by various forms other than the flap. Further, in the present embodiment, the cornea is cut off by using the cutting-off beam or the cornea can be cut off by using a device such as a microkeratome.

When the cornea 2 is cut off and the flap 3 is obtained, the vision correction beam 6 is radiated to the stromal bed 4 that has been covered by the flap 3 (S300). The vision correction beam 8 radiated to the stromal bed 4 of the eyeball 1 can make the stromal bed 4 flat and can change the curvature of the stromal bed 4.

When the vision correction step ends, a step of welding the cut cornea is performed (S400). In order to perform the present step, the flap separated from the cornea is disposed at the cut position of the cornea again. In addition, the step is performed by determining the welding position and irradiating the welding beam to the determined welding position.

First, in the step of the determining the welding position, the welding position is determined based on the image information obtained from the aforementioned image unit 13. The control unit may use the obtained image information while performing the cutting-off step and/or the vision correction step or use the image photographed while the flap is disposed at the cut position again. Based on this, patterns and respective horizontal coordinates and depth coordinates which are irradiated with a plurality of welding beams may be determined. In this case, the horizontal coordinate which is irradiated with the welding beam may correspond to the cut position of the cornea, that is, the edge of the flap. In addition, the depth coordinate (vertical coordinate) which is irradiated with the welding beam may be a depth corresponding to a size of 20% to 80% of the thickness of the flap from the corneal surface.

When the position at which the welding beam is irradiated is determined, the control unit drives the welding beam generating apparatus and the beam delivery apparatus to irradiate the welding beam to a target position of the cornea. In this case, the welding beam is irradiated to the inner side with a predetermined depth of the cornea in a converged state. In addition, the welding beams are sequentially irradiated to a plurality of positions according to the set irradiation pattern.

A spot size of the welding beam may be a size of 0.1 μm to 10 μm. A pulse width of the welding beam may be a width of 1 μs to 1 s. An output of the welding beam is in a range of 1 μJ to 100 μJ. As one example, the welding beam of this embodiment may have a wavelength of 1319 nm, a pulse width of 1 ms and an output of 40 μJ. However, as an example, the welding beam can have various parameter other than the abovementioned parameters.

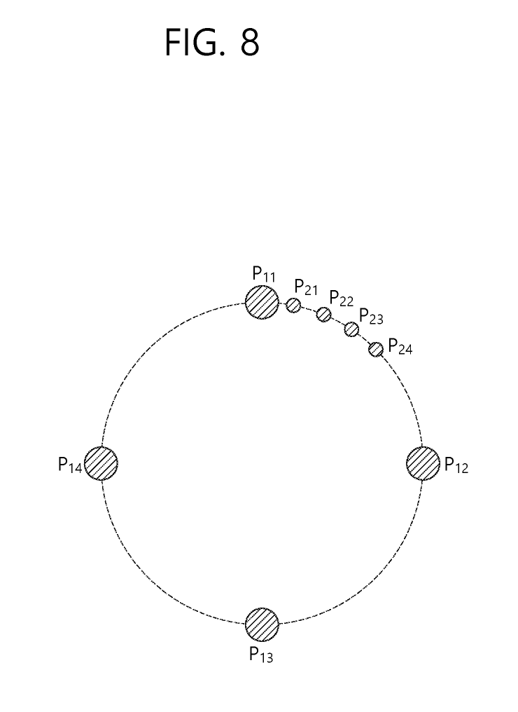

FIG. 8 is a diagram illustrating an example of a pattern irradiated with the welding beam. The welding beam is irradiated to the welding position (corresponding to the cut position in the cutting-off step) formed along the edge of the flap. In FIG. 8, the pattern is a pattern formed by irradiating secondary welding beams P21 to P2n) at tight intervals after irradiating primary welding beams P11 to P14 at wide intervals according to a welding position. The primary welding beam is a beam which is preliminarily irradiated to perform the welding step while the flap is temporarily fixed on the cornea and may be irradiated with a size larger than that of the secondary welding beam or with a high output. In addition, while the position of the flap is fixed somewhat by the primary welding beams, the secondary welding beams that perform the full-scale welding may be sequentially irradiated. However, FIG. 8 is just an example, and the secondary welding beams may also be sequentially irradiated clockwise along the circumference of the flap edge.

Accordingly, it is possible to prevent the flam from being separated by an external impact by welding the cut portion between the cornea and the flap separated from the cornea with the welding beam, and thus it is possible to the patient's satisfaction at the operation.

Although embodiments of the present invention were described above with reference to the accompanying drawings, those skilled in the art would understand that the present invention may be implemented in various ways without changing the necessary features or the spirit of the prevent invention. Therefore, the embodiments described above are only examples and should not be construed as being limitative in all respects. The scope of the present invention is defined by not the specification, but the following claims, and all of changes and modifications obtained from the meaning and range of claims and equivalent concepts should be construed as being included in the scope of the present invention.

What is claimed is:

1. An ophthalmic surgery apparatus comprising:
a cutting-off beam generating unit that generates a cutting-off beam for cutting off a part of the cornea for a vision correction surgery;
a welding beam generating unit that generates a welding beam having a wavelength in a near-infrared band for welding a part of the cut cornea by irradiating the welding beam to the cut position of the cornea;
a beam delivery unit that delivers the cutting-off beam and the welding beam to the cut position of the cornea;
an image unit that obtains image information on the cut position of the cornea; and
a control unit that controls an irradiation position of the welding beam based on the cut position information of the cornea obtained by the image unit.

2. The ophthalmic surgery apparatus of claim 1, wherein the welding beam has a wavelength of 800 nm to 980 nm or 1150 nm to 1390 nm.

3. The ophthalmic surgery apparatus of claim 1, wherein the welding beam has a wavelength of 840 nm to 950 nm or 1160 nm to 1370 nm.

4. The ophthalmic surgery apparatus of claim 1, wherein the welding beam has a wavelength in a band of which an absorption coefficient to water is 10 or less and an absorption coefficient to collagen is 0.1 or more.

5. The ophthalmic surgery apparatus of claim 1, wherein a part of the cornea is cut off in a flap form having a predetermined thickness and the welding beam is irradiated to be focused to a target position which is positioned at a predetermined depth along the edge of the flap, and the target position is positioned at a depth corresponding to 0.2 to 0.8 of the flap thickness from the surface of the cornea.

6. The ophthalmic surgery apparatus of claim 5, wherein the welding beam is a femtosecond laser.

7. An ophthalmic surgery apparatus comprising:
a welding beam generating unit that generates a welding beam having a wavelength in a near-infrared band and irradiated to a cut position of the cornea to weld the cut position; and
a beam delivery unit that delivers the welding beam to the cut position of the cornea.

8. The ophthalmic surgery apparatus of claim 7, wherein the welding beam has a wavelength in a band of which an absorption coefficient to water is 10 or less and an absorption coefficient to collagen is 0.1 or more.

9. The ophthalmic surgery apparatus of claim 7, wherein the welding beam has a wavelength of 800 nm to 980 nm or 1150 nm to 1390 nm.

10. An ophthalmic surgery method comprising the steps of:
cutting off a part of the cornea by irradiating a cutting-off beam before performing a vision correction treatment; and
welding the cornea by irradiating a welding beam having a wavelength in a near-infrared band to the cut position of the cornea.

11. The ophthalmic surgery method of claim 10, wherein the welding beam has a wavelength of 800 nm to 980 nm or 1150 nm to 1390 nm.

12. The ophthalmic surgery method of claim 10, wherein the welding beam has a wavelength in a band of which an absorption coefficient to water is 10 or less and an absorption coefficient to collagen is 0.1 or more.

13. The ophthalmic surgery method of claim 10, wherein the step of welding the cornea includes the steps of
determining a position irradiated by the welding beam based on image information obtained by the image unit and
irradiating the welding beam according to the determined welding position.

14. The ophthalmic surgery method of claim 13, wherein the welding beam is irradiated along the boundary of the flap of the cut cornea in the step of cutting off the part of the cornea and focused at a depth corresponding to 0.2 to 0.8 of the flap thickness of the cornea from the corneal surface.
